(19) United States
(12) Patent Application Publication
(10) Pub. No.: US 2026/0159468 A1
(43) Pub. Date: Jun. 11, 2026

(54) PROCESS FOR BORANE-MEDIATED ESTER REDUCTION

(71) Applicant: Provivi, Inc., Santa Monica, CA (US)

(72) Inventors: Konrad OTTE, Los Angeles, CA (US); Micah SHEPPARD, Los Angeles, CA (US); Jozsef REPASI, Budapest (HU); Andras SZABO, Budapest (HU)

(73) Assignee: Provivi, Inc., Santa Monica, CA (US)

(21) Appl. No.: 18/718,366

(22) PCT Filed: Dec. 15, 2022
(86) PCT No.: PCT/US2022/053083

§ 371 Date: Jun. 10, 2024

Related U.S. Application Data
(60) Provisional application No. 63/290,614, filed on Dec. 16, 2021.

(51) Int. Cl. C07C 45/54; C07C 45/82

(52) U.S. Cl. CPC C07C 45/54; C07C 45/82

(57) ABSTRACT

Methods for preparation of aldehydes from carboxylic acid esters, including unsaturated fatty acid esters and triacyl glycerides, are described. The methods include combining an ester with a silane reagent and a borane catalyst to form a mixed silyl acetal which is hydrolyzed to provide the aldehyde product. The reagents are combined in a controlled manner to limit the generation of heat and hydrogen gas. New procedures for hydrolysis of acetal intermediates and improved purification and recovery processes provide aldehyde products in excellent yield.

### CROSS-REFERENCE TO RELATED APPLICATIONS

The present application claims priority to U.S. Provisional Pat. Appl. No. 63/290,614, filed on Dec. 16, 2021, which application is incorporated herein by reference in its entirety.

### BACKGROUND

The reduction of esters to aldehydes is an important transformation in organic chemistry and numerous preparative methods have been developed for this purpose. Boron-based Lewis acids catalyze the reduction of esters by silane reagents as described, for example, by Parks et al. (J. Org. Chem., 2000, 65, 3090-3098). The reduction is exothermic and requires activation of the catalyst. Upon mixing all components together there is a strong uncontrolled hydrogen formation, caused by protic impurities in the feedstocks. Only after cessation of the gas formation, the catalyst is activated, and the reaction starts resulting in an uncontrolled exotherm. During gas formation there is no reaction observed, likewise this phase is not accompanied by a noticeable exotherm. Both effects, the gas formation and the exotherm, are problematic, certainly detrimental, and potentially dangerous on scale if left uncontrolled.

### BRIEF SUMMARY

Provided herein are methods for preparing an aldehyde product. The methods include:
forming a reaction mixture comprising a silane, a borane catalyst, and an ester according to Formula III:

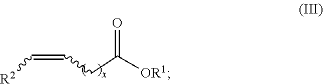

maintaining the reaction mixture under conditions for forming a mixed acetal according to Formula II:

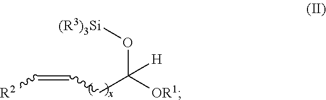

and
converting the mixed acetal to an aldehyde according to Formula I:

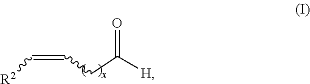

thereby forming the aldehyde product;
wherein:
subscript x is an integer ranging from 0 to 17,
subscript y is an integer ranging from 0 to 17,
R¹ is C₁₋₈ alkyl or glycerol,
R² is selected from the group consisting of C₁₋₁₈ alkyl and C₁₋₁₈ alkenyl, and
each R³ is independently selected from the group consisting of C₁₋₈ alkyl and C₆₋₁₄ aryl.

In some embodiments, the aldehyde product is an aldehyde according to Formula Ia:

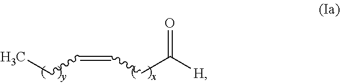

wherein subscript y is an integer ranging from 0 to 17.

Methods according to the present disclosure are conducted so as to mitigate and control the detrimental and potentially dangerous generation of heat and hydrogen gas during reduction of the ester starting material.

### BRIEF DESCRIPTION OF THE DRAWINGS

FIG. 1 shows an exemplary embodiment of fatty acid ester reduction and subsequent work-up according to the present disclosure, employing borane catalysts and stoichiometric amounts of silane reagents to form a mixed silyl acetal intermediate. The intermediate is hydrolyzed to afford the desired aldehyde product. Methyl (Z)-hexadec-11-enoate (Z11-16ME) is depicted for simplicity, although other esters and mixtures of esters can be employed.

FIG. 2 shows an alternative embodiment including conversion of the mixed silyl acetal to a dialkyl acetal.

### DETAILED DESCRIPTION

The present disclosure provides scalable processes for the preparation of unsaturated fatty aldehydes. The aldehydes can be used, for example, as technical grade active ingredients in pheromone formulations for insect mating disruption in agricultural applications. The new processes include controlled activation of borane catalysts for reduction of esters, providing improved safety during implementation. Reaction selectivity can be increased by controlling the reaction exotherm, thereby avoiding unwanted over-reduction. Mild hydrolysis of acetal intermediates (e.g., using acetic acid or another short-chain carboxylic acid) and mild removal of silane impurities (e.g., via steam distillation) provide the desired products without major degradation of sensitive aldehyde functional groups.

### I. Definitions

As used herein, the term “silane” refers to a compound containing one or more silicon atoms bonded to four independently-selected substituents, Si(R)₄. Typically, at least one of the substituents is hydrogen. An alkyl silane refers to a silane wherein at least one of the four substituents is an alkyl group. Silanes include compounds according to the formula SiₙR₂ₙ₊₂ or the formula SiₙR₂ₙ₊₃, wherein subscript n is an integer ranging from 1 to 10. Silanes according to the formula SiₙR₂ₙ₊₃, wherein one R is an oxygen atom bridging two silicon atoms, are also referred to as siloxanes.

As used herein, the term “borane” refers to a compound containing a boron atom bonded to three independently-selected substituents, B(R)₃.

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups can be substituted with one or more groups selected from halo, hydroxy, amino, alkylamino, amido, acyl, nitro, cyano, and alkoxy.

As used herein, the term “alkenyl” refers to an alkyl group, as defined herein, having one or more carbon-carbon double bonds. Alkenyl groups include “dienyl” groups having two carbon-carbon double bonds and “trienyl” groups having three carbon-carbon double bonds. Alkenyl groups also include “enynyl” groups having an alkynyl moiety in addition to an alkenyl moiety (i.e., having at least one carbon-carbon double bond and at least one carbon-carbon triple bond).

As used herein, the term “alkynyl” refers to an alkyl group, as defined herein, having one or more carbon-carbon triple bonds.

As used herein, the term “cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. Unless otherwise specified, “substituted cycloalkyl” groups can be substituted with one or more groups selected from halo, hydroxy, amino, alkylamino, amido, acyl, nitro, cyano, and alkoxy. The term “lower cycloalkyl” refers to a cycloalkyl radical having from three to seven carbons including, for example, cyclopropyl, cyclobutyl, cyclopentyl, cyclohexyl, and cycloheptyl.

As used herein, the terms “halo” and “halogen,” by themselves or as part of another substituent, refer to a fluorine, chlorine, bromine, or iodine atom.

As used herein, the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. Unless otherwise specified, “substituted aryl” groups can be substituted with one or more groups selected from halo, hydroxy, amino, alkylamino, amido, acyl, nitro, cyano, and alkoxy.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms can be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups can be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. Unless otherwise specified, “substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

As used herein, the term “amino” refers to a moiety —NR₂, wherein each R group is H or alkyl. An amino moiety can be ionized to form the corresponding ammonium cation.

As used herein, the term “sulfonyl” refers to a moiety —SO₂R, wherein the R group is alkyl, haloalkyl, or aryl. An amino moiety can be ionized to form the corresponding ammonium cation. “Alkylsulfonyl” refers to an amino moiety wherein the R group is alkyl.

As used herein, the term “hydroxy” refers to the moiety —OH.

As used herein, the term “cyano” refers to a carbon atom triple-bonded to a nitrogen atom (i.e., the moiety —C—N).

As used herein, the term “carboxy” refers to the moiety —C(O)OH. A carboxy moiety can be ionized to form the corresponding carboxylate anion.

As used herein, the term “amido” refers to a moiety —NRC(O)R or —C(O)NR₂, wherein each R group is H or alkyl.

As used herein, the term “nitro” refers to the moiety —NO₂.

As used herein, the terms “forming a reaction mixture” and “contacting” refer to the process of bringing into contact at least two distinct species such that they can react. It should be appreciated, however, that the resulting reaction product can be produced directly from a reaction between the added reagents or from an intermediate from one or more of the added reagents that can be produced in the reaction mixture.

The terms “about” and “around,” as used herein to modify a numerical value, indicate a close range surrounding that explicit value. If “X” were the value, “about X” or “around X” would indicate a value from 0.9× to 1.1×, and in certain instances, a value from 0.95× to 1.05× or from 0.98× to 1.02×, or from 0.99× to 1.01×. Any reference to “about X” or “around X” specifically indicates at least the values X, 0.95×, 0.96×, 0.97×, 0.98×, 0.99×, 1.01×, 1.02×, 1.03×, 1.04×, and 1.05×. Thus, “about X” and “around X” are intended to teach and provide written description support for a claim limitation of, e.g., “0.99×.”

### II. Methods for the Preparation of Aldehyde Products

Provided herein are methods for preparing an aldehyde product. The methods include:
forming a reaction mixture comprising a silane, a borane catalyst, and an ester according to Formula III:

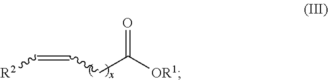

maintaining the reaction mixture under conditions for forming a mixed acetal according to Formula II:

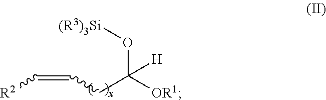

and
converting the mixed acetal to an aldehyde according to Formula I:

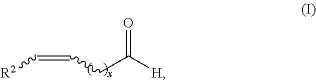

thereby forming the aldehyde product;
wherein:
subscript x is an integer ranging from 0 to 17,
R¹ is C₁₋₈ alkyl or glycerol,
R² is selected from the group consisting of C₁₋₁₈ alkyl and C₁₋₁₈ alkenyl, and
each R³ is independently selected from the group consisting of C₁₋₈ alkyl and C₆₋₁₄ aryl.

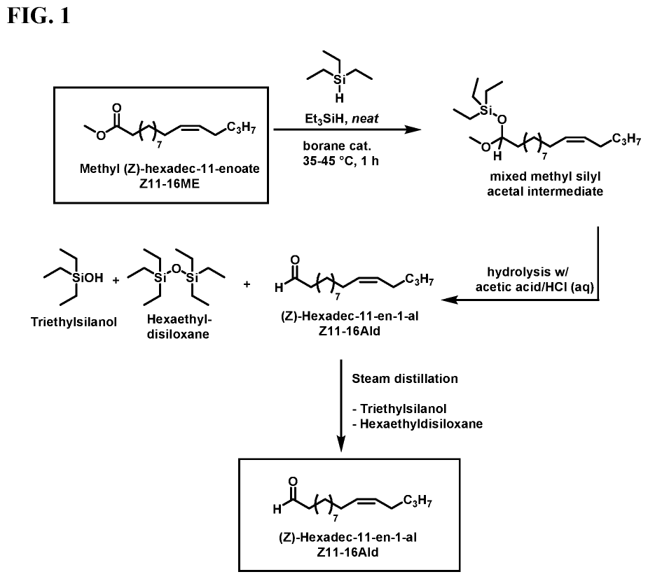

FIG. 1 shows a non-limiting example of a process according to the present disclosure, from formation of a mixed silyl acetal via reduction of an ester starting material, to hydrolysis of the acetal, to purification of the final aldehyde blend. Methyl (Z)-hexadec-11-enoate (Z11-16ME) is depicted in FIG. 1, which may be present as a mixture of other fatty acid methyl esters (FAMES, e.g., methyl (Z)-hexadec-9-enoate (Z9-16ME); (Z)-octadec-9-enoate (Z9-18ME); methyl (Z)-octadec-13-enoate (Z13-18ME); and the like). The process can also be employed more generally for the direct reduction of any suitable ester, for example triacyl glycerides as in soybean oil, to the corresponding aldehyde. In a first step, the ester starting material is reduced in a set temperature range to form a mixed acetal intermediate. This intermediate is then hydrolyzed using an aqueous acid to provide a crude aldehyde, which may include impurities such as silanols and disiloxanes. These impurities may be selectively removed via distillation (e.g., steam distillation). These impurities may alternatively be selectively removed via vacuum distillation.

In some embodiments, R² is C₁₋₁₈ alkyl. In some embodiments, for example, the ester is an ester according to Formula IIIa or a triacyl glyceride (TAG) according to Formula IIIc:

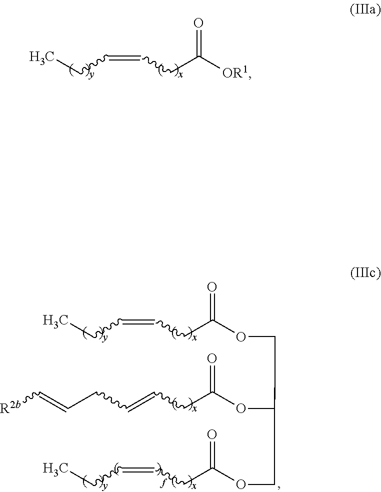

wherein f is 0 or 1. The mixed acetal is a mixed acetal according to Formula IIa:

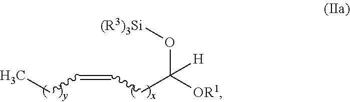

and
the aldehyde product is an aldehyde according to Formula Ia:

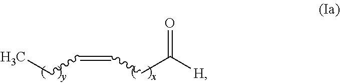

wherein subscript y is an integer ranging from 0 to 17.

In some embodiments, R² is C₁₋₁₈ alkenyl. For example, the ester can be an ester according to Formula IIIb:

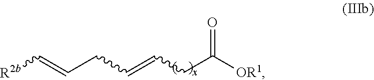

the mixed acetal can be a mixed acetal according to Formula IIb:

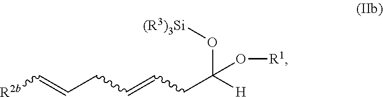

and
the aldehyde product can be an aldehyde according to Formula Ib:

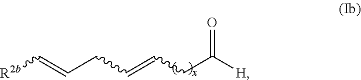

wherein R^{2b} is C₁₋₁₄ alkyl. The aldehyde according to Formula Ib may be, for example, (Z,Z)-9,12-octadecadienal wherein subscript x is 7.

A number of silane reagents are suitable for use in the methods according to the present disclosure. Examples of such silanes include, but are not limited to, alkylsilanes (e.g., triethylsilane, diethylsilane, triisopropylsilane, and the like), alkylsiloxanes (e.g., polymethylhydrosiloxane, diethoxy-methylsilane, triethoxysilane, and the like), phenylsilanes (e.g., phenysilane, diphenylsilane, triphenylsilane, and the like), trichlorosilane, and tris(trimethylsilyl)silane. In some embodiments, the silane is an alkylsilane. In some embodiments, the silane is triethylsilane.

In some embodiments, the borane catalyst is a compound according to Formula IV:

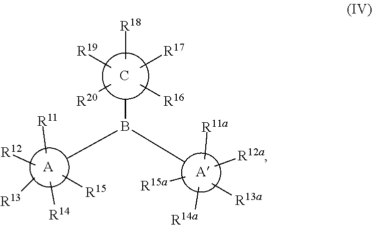

wherein
B is boron;
the A ring and the A′ ring are aryl groups; wherein
R¹¹ and R^{11a} are independently selected from groups having small steric demand, preferably H, D and F;
R¹⁵ and R^{15a} are independently selected from groups having small steric demand, preferably H, D and F;
R¹², R¹³, R¹⁴, R^{12a}, R^{13a} and R^{14a} are independently selected from the group consisting of H, D, F, Cl, Br, I, SF₅, alkyl, cycloalkyl, alkenyl, cycloalkenyl, aryl and heteroaryl, wherein alkyl, cycloalkyl, alkenyl, cycloalkenyl, aryl, and heteroaryl are optionally substituted;
the C ring is aryl group, wherein
R¹⁶ is selected from groups having small steric demand, preferably H, D and F;
R²⁰ is selected from groups having large steric demand, preferably from the group consisting of Cl, Br, I, SF₅, alkyl, cycloalkyl, alkenyl, cycloalkenyl, aryl and heteroaryl, wherein alkyl, cycloalkyl, alkenyl, cycloalkenyl, aryl and heteroaryl are optionally substituted;
R¹⁷, R¹⁸ and R¹⁹ are independently selected from the group consisting of H, D, F, Cl, Br, I, SF₅, alkyl, cycloalkyl, alkenyl, cycloalkenyl, aryl and heteroaryl, where alkyl, cycloalkyl, alkenyl, cycloalkenyl, aryl and heteroaryl are optionally substituted. In some embodiments, R²⁰ is not a pentafluorophenyl group when R¹¹-R¹⁵, R^{11a}-R^{15a}, and R¹⁶-R¹⁹ are F.

In some embodiments, the catalyst has a structure according to Formula Iva:

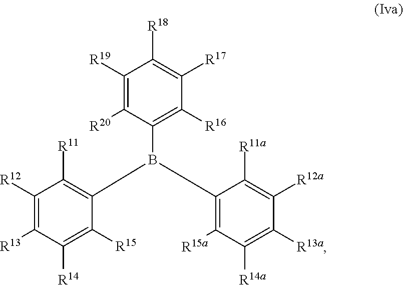

wherein
R¹¹ and R^{11a} are independently selected from the group consisting of H, D and F;
R¹⁵ and R^{15a} are independently selected from the group consisting of H, D and F;
R¹², R¹³, R¹⁴, R^{12a}, R^{13a} and R^{14a} are independently selected from the group consisting of H, D, F, Cl, Br, alkyl, cycloalkyl and aryl, wherein alkyl, cycloalkyl and aryl are optionally substituted;
R¹⁶ is selected from the group consisting of H, D and F;
R²⁰ is selected from the group consisting of Cl, Br, I, SF₅, alkyl, cycloalkyl and aryl, wherein alkyl, cycloalkyl and aryl are optionally substituted;
R₇, R₈ and R₉ are independently selected from the group consisting of H, D, F, Cl, Br, alkyl and cycloalkyl, wherein alkyl and cycloalkyl are optionally substituted.

In some embodiments:
R¹¹, R^{11a}, R¹⁵ and R^{15a} are F;
R¹², R¹³, R¹⁴, R^{12a}, R^{13a}, and R^{14a} are independently selected from H and F,
R¹⁶, R¹⁷, R¹⁸ and R¹⁹ are independently selected from H and F; and
R²⁰ is selected from C₁, Br, and pentafluorophenyl.

In some embodiments, the A ring and the A′ ring are independently selected from the group consisting of pentafluorophenyl, 2,3,4,6-tetrafluorophenyl, 2,4,6-trifluorophenyl, and 2,3,6-trifluorophenyl.

In some embodiments, the C ring is selected from the group consisting of 2-chloro-6-fluorophenyl, 2-bromo-6-fluorophenyl, and perfluoro-1,1′-biphen-2-yl.

In some embodiments, the catalyst is selected from the group consisting of:

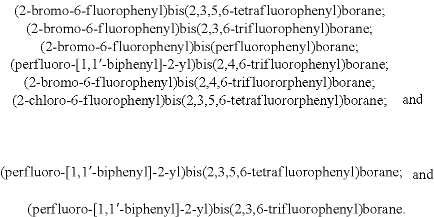

In some embodiments, the catalyst is selected from the group consisting of:

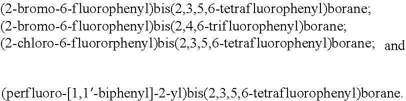

In some embodiments, the catalyst is (2-bromo-6-fluorophenyl)bis(2,3,5,6-tetrafluorophenyl)borane.

As described above, combination of starting material, catalyst, and silane results in strong, uncontrolled formation of hydrogen caused by protic impurities that are typically present in silane feedstocks. In the new processes provided herein, this dangerous behavior is controlled by initiating the reaction with a portion of the reactants to activate the catalyst without excessive gas formation and heat formation. For example, a first portion of the borane catalyst may be combined with a mixture of the ester and the silane, or a first amount of the silane reagent and/or the ester starting material in combination with the borane catalyst.

In this manner, the catalyst can be activated without excessive gas formation and heat generation. Upon catalyst activation and cessation of gas formation, the remaining reactants can be safely added. Gas formation occurs as the reaction progresses using this approach, and both the exotherm and the gas formation can be controlled by adjusting the flow speed of the substrate and reagent addition. In some instances, the entire amount of the ester starting material can be employed in the early activation phase with portion of the silane. Upon activation of the catalyst and cessation of the gas formation, the remainder of the silane reagent can be added in a controlled way. Such instances are advantageous in that only one component stream needs be managed instead of two.

Accordingly, forming the reaction mixture in some embodiments of the present disclosure includes: (a) combining the ester with the silane; and (b) adding the borane catalyst to the mixture resulting from step (a). The catalyst made be added in small discrete portions (e.g., dropwise) or in continuous fashion over a time period ranging from a few minutes to several hours.

In some embodiments, forming the reaction mixture includes combining the ester starting material with a first portion of the silane to form a first mixture. Typically, the ester starting material will be combined with up to about 25% of the total silane in the initial stage of the process. The ester may be combined, for example, with about 1-25%, 1-20%, 5-20%, 5-15%, 8-12%, or 9-11% of the total silane. In some embodiments, about 1, 2, 3, 4, 5, 6, 7, 8, 9, 10, 11, 12, 13, 14, 15, 16, 17, 18, 19, or 20% of the total silane is combined with the ester in the first mixture. In some embodiments, the first portion of the silane constitutes from about 5% to about 15% of the total silane.

In some embodiments, a first portion of the ester according to Formula III (e.g., up to about 25% of the total amount of the ester) is combined the first portion of silane in the first mixture. The first portion of the silane may be combined, for example, with about 1-25%, 1-20%, 5-20%, 5-15%, 8-12%, or 9-11% of the total ester. In some embodiments, about 1, 2, 3, 4, 5, 6, 7, 8, 9, 10, 11, 12, 13, 14, 15, 16, 17, 18, 19, or 20% of the total ester is combined with the first portion of the silane in the first mixture. In some embodiments, the first portion of the ester constitutes from about 5% to about 15% of the total ester. Greater amounts of the ester, including up to essentially all of the ester (i.e., 100% of the ester) may also be combined with the first portion of silane in the first mixture.

Following formation of the first mixture, the borane catalyst is added in one or more portions to form a second mixture. In some embodiments, substantially all of the borane catalyst is added in one portion to form the second mixture. Typically, the methods further include adding a second portion of the silane to the second mixture. When only a first portion of the ester is included in the first mixture, the methods typically include adding a second portion of the ester to the second mixture. The second portion of the ester may be added to the second mixture concomitantly with the second portion of the silane, or the reagents may be added in stepwise fashion. In some embodiments, the second portion of the silane and/or the ester constitutes the balance of the total amount. For example, the second portion of the silane and/or the ester may constitute 90% of the total amount when the first portion of the silane and/or the ester constitutes 10% of the total amount. In some embodiments, 75-99%, 80-99%, 80-95%, 85-95%, 88-92%, or 89-91% of the total silane and/or ester is added in the second portion. In some embodiments, about 80, 81, 82, 83, 84, 85, 86, 87, 88, 89, 90, 91, 92, 93, 94, 95, 96, 97, 98, or 99% of the total silane and/or ester is added in the second portion. The total amount of silane and/or ester may added as more than two portions if appropriate, e.g., if necessary for controlling gas formation or heat generation. For example, 10 or more portions may be used to add the total amount of silane and/or ester to the reaction mixture. Multiple portions may be added as discrete amounts, or the silane and/or the ester may be introduced continuously at rates controlled to limit gas formation and/or heat generation.

In some embodiments, the ester, the silane, and the borane catalyst constitute the entirety of the reaction mixture or substantially all of the reaction mixture. Alternatively, the reaction mixture may further contain on ore more solvents such as C₅-C₂ alkanes, C₃-C₈ cycloalkanes, alkyl esters, aryl esters, aromatic hydrocarbons, ketones, ethers, water, and combinations thereof. Examples of suitable solvents include, but are not limited to, pentane, hexane, heptane, octane, cyclopentane, cyclohexane, cycloheptane, ethyl acetate, isopropyl acetate, toluene, xylene, cumene, 1,2,4-trimethylbenzene, acetone, butanone, pentanone, methyl isopropyl ketone, methyl isobutyl ketone, tetrahydrofuran, methyl tert-butyl ether (MTBE), methoxymethane, ethoxyethane, and the like.

Any suitable amount of the silane can be used in the methods according to the present disclosure. In general, the total amount of silane in the reaction mixture after addition of all portions ranges from about 1 to about 2.5 molar equivalents of the silane with respect to the ester according to Formula III. For example, the reaction mixture can contain from about 1 to about 1.25 equivalents, or from about 1.25 to about 1.5 equivalents, or from about 1.5 to about 1.75 equivalents, or from about 1.75 to about 2 equivalents, or from about 2 to about 2.25 equivalents, or from about 2.25 to about 2.5 equivalents of the silane with respect to the ester.

The borane catalyst is generally employed in sub-stoichiometric amounts, typically in the range of 0.005 mol % to 10 mol % with respect to the ester starting material. The reaction mixture may contain, for example, from about 0.005 to about 7.5 mol %, or from about 0.01 to about 5 mol %, or from about 0.1 to about 5 mol %, or from about 0.25 to about 2.5 mol %, or from about 0.75 mol % to about 1.25 mol % borane catalyst. In some embodiments, the reaction mixture contains 0.01, 0.025, 0.05, 0.1, 0.2, 0.3, 0.4, 0.5, 0.6, 0.7, 0.8, 0.9, 1.0, 1.1, 1.2, 1.3, 1.4, or 1.5 mol % borane catalyst.

During the reduction, it is possible to over-reduce the aldehyde product to alcohol. In other known processes, over-reduction is avoided by lowering the reaction temperature. It has now been discovered, however, that over-reduction can be minimized in the methods of the present disclosure by raising the reaction temperature. In some instances, for example, the level of over-reduction may be decreased by 30% or more when the reaction temperature is raised from 20° C. to 40° C. In some embodiments, the reaction mixture (containing any amount of ester, silane, and borane catalyst, at any stage of combination/addition) is maintained at a temperature ranging from about 35° C. to about 45° C. (e.g., from 35° C. to 38° C., or from 38° C. to 42° C., or from 42° C. to 45° C.). Catalyst inactivation may occur at higher temperatures, leading to decreases in yield by 10% or greater, so the reaction mixture is usually maintained at no more than about 60° C. In some embodiments, the reaction mixture is maintained as a temperature less than 55° C., or less than 50° C. The reaction mixture can be maintained under the reaction conditions for any period of time sufficient to form the mixed acetal according to Formula II. In general, the reaction mixture is maintained under the reaction conditions for a period of time ranging from a few minutes to a few hours or longer, depending in part on factors such as the specific catalyst employed.

The desired aldehyde product is not formed directly but rather as a mixed silyl acetal, as shown in FIG. 1. The mixed acetal can be converted to the final aldehyde product via a hydrolysis reaction, e.g., an aqueous acid-promoted hydrolysis. Depending on factors such as the specific acid or the structure of the particular acetal, the reaction may be limited by: low water solubility and limited transfer of the mixed acetal into the aqueous phase; use of cosolvents that tend to complicate recovery of the desired aldehyde products and that may undergo acid-catalyzed side reactions; and/or susceptibility of the aldehyde to degradation under harsh acidic conditions. If necessary, such problems can be circumvented by using a short-chain carboxylic acid such as acetic acid, formic acid, or propionic acid as a solvent for the hydrolysis. Using various amounts of diluted hydrochloric acid, the mixed acetal can be hydrolyzed under mild conditions, ensuring stability of the aldehyde and providing easy extraction of the crude aldehyde from the hydrolysis mixture. The acetic acid/aqueous hydrochloric acid mixture can be recycled and losses of aldehyde to the aqueous phase are kept to a minimum. In some embodiments, converting the mixed acetal to the aldehyde comprises hydrolyzing the mixed acetal. In some embodiments, hydrolyzing the mixed acetal or the dialkyl acetal comprises combining the mixed acetal or the dialkyl acetal with acetic acid and hydrochloric acid. The acetic acid and hydrochloric acid may be employed in varying ratios, e.g., 1-5 volumes of acetic acid combined with 1-5 volumes of 0.25-5 M aqueous hydrochloric acid. In some embodiments, 4 volumes of acetic acid are employed in combination with one volume of 1 M aqueous hydrochloric acid.

During hydrolysis, trialkylsilanols and hexaalkyldisiloxanes are formed as the two major byproducts. Trialkylsilanes (e.g., triethylsilanol) are formed upon hydrolysis of the mixed acetal and are prone to condensation to hexaalkyldisiloxanes (e.g., hexaethyldisiloxane) under acidic conditions. Both byproducts accumulate in the organic phase and are not removable by extraction. The crude product cannot be distilled by regular vacuum distillation because of the stability limitations of the aldehyde product. Instead of resorting to expensive separation techniques, such as chromatography, purification can be accomplished via steam distillation. Using this technique, the silicon-containing impurities can be removed to less than 1% and the aldehydes are obtained in high yield. The steam distillation can be operated in batch mode or continuous mode, under ambient pressure or under reduced pressure (e.g., around 200 mbar). Compared to regular vacuum distillation, exposure of the sensitive aldehyde to heat is minimized.

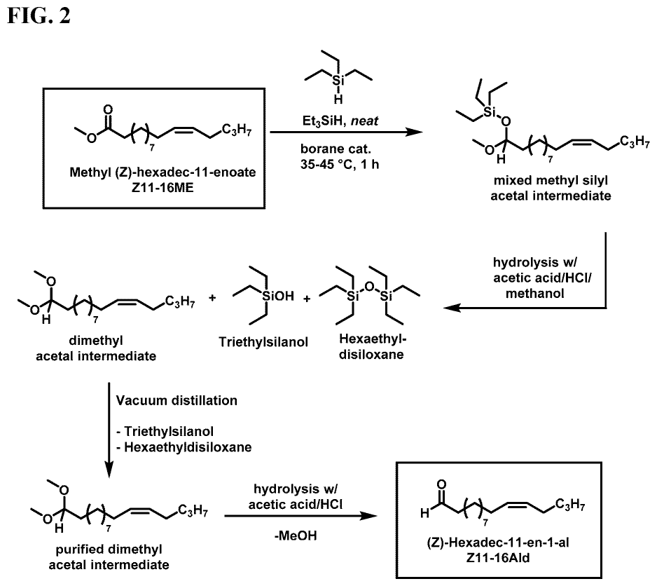

Alternatively, a different reaction and purification sequence can be conducted as shown in FIG. 2. Instead of hydrolyzing the mixed aldehyde directly, it can first be converted into a dialkyl acetal (e.g., a dimethyl acetal). Purification by vacuum distillation is possible with dialkyl acetals because they are generally more stable than the mixed acetals and significantly more stable than the free aldehydes. After distillation and removal of the major byproducts, the dialkyl acetal is hydrolyzed to provide the final aldehyde. The benefits of this approach include a simplified vacuum distillation to remove byproducts and the option for longer-term storage of the dimethyl acetal intermediate.

In some embodiments, the methods further include removing silanol and disiloxane byproducts produced during hydrolysis of the mixed acetal. In some embodiments, the byproducts are removed via vacuum distillation or steam distillation.

In some embodiments, converting the mixed acetal to the aldehyde comprises converting the mixed acetal to a dialkyl acetal and hydrolyzing the dialkyl acetal. In some embodiments, hydrolyzing the dialkyl acetal comprises combining the mixed acetal or the dialkyl acetal with acetic acid and hydrochloric acid.

A number of aldehyde products can be prepared according to the methods provided herein. In some embodiments, the compound according to Formula I is selected from E-2-decenal; (Z)-2-decenal; (Z)-4-decenal; (Z)-5-decenal; (E,E)-2,4-decadienal; (E,Z)-2,4-decadienal; (Z,Z)-2,4-decadienal, (E)-2-undecenal; (E)-2-dodecenal; (Z)-5-dodecenal; (E)-6-dodecenal; (E)-7-dodecenal; (Z)-7-dodecenal; (E)-8-dodecenal; (E)-9-dodecenal; (Z)-9-dodecenal; (E)-10-dodecenal; (E,Z)-5,7-dodecadienal; (Z,E)-5,7-dodecadienal; (Z,Z)-5,7-dodecadienal; (E,Z)-7,9-dodecadienal; (E,E)-8,10-dodecadienal; (E,Z)-8,10-dodecadienal; (Z,E)-8,10-dodecadienal; (Z)-4-tridecenal; (E)-5-tetradecenal; (Z)-5-tetradecenal; (Z)-7-tetradecenal; (Z)-8-tetradecenal; (E)-11-tetradecenal; (Z)-11-tetradecenal; (E,E)-2,4-tetradecadienal; (E,Z)-4,9-tetradecadienal; (E,E)-5,8-tetradecadienal; (Z,Z)-5,8-tetradecadienal; (E,E)-8,10-tetradecadienal; (E,Z)-8,10-tetradecadienal; (Z,Z)-8,10-tetradecadienal; (Z,E)-9,11-tetradecadienal; (Z,Z)-9,11-tetradecadienal; (Z,E)-9,12-tetradecadienal; (E,E)-10,12-tetradecadienal; (Z)-10-pentadecenal; (Z,Z)-6,9-pentadecadienal; (E,Z)-9,11-pentadecadienal; (Z,Z)-9,11-pentadecadienal; (Z)-9-hexadecenal; (E)-10-hexadecenal; (Z)-10-hexadecenal; (E)-11-hexadecenal; (Z)-11-hexadecenal, (Z)-12-hexadecenal; (E)-14-hexadecenal; (E)-7-hexadecenal; (Z)-7-hexadecenal; (E)-9-hexadecenal; (E,Z)-4,6-hexadecadienal; (E,Z)-6,11-hexadecadienal; (Z,E)-7,11-hexadecadienal; (Z,Z)-7,11-hexadecadienal; (E,Z)-8,11-hexadecadienal; (E,E)-9,11-hexadecadienal; (E,Z)-9,11-hexadecadienal; (Z,E)-9,11-hexadecadienal; (Z,Z)-9,11-hexadecadienal; (E,E)-10,12-hexadecadienal; (E,Z)-10,12-hexadecadienal; (Z,E)-10,12-hexadecadienal; (Z,Z)-10,12-hexadecadienal; (E,E)-11,13-hexadecadienal; (E,Z)-11,13-hexadecadienal; (Z,E)-11,13-hexadecadienal; (Z,Z)-11,13-hexadecadienal; (E,E)-10,14-hexadecadienal; (E,E,Z)-4,6,11-hexadecatrienal; (E,E,E)-10,12,14-hexadecatrienal; (E,E,Z)-10,12,14-hexadecatrienal; (E,E,Z)-4,6,11,13-hexadecatetraenal; (E)-2-heptadecenal; (Z)-heptadecenal; (Z)-9-heptadecenal; (E)-2-octadecenal; (E)-9-octadecenal; (Z)-9-octadecenal; (E)-11-octadecenal; (Z)-11-octadecenal; (E)-13-octadecenal; (Z)-13-octadecenal; (E)-14-octadecenal; (E,Z)-2,13-octadecadienal; (E,Z)-3,13-octadecadienal; (Z,Z)-3,13-octadecadienal; (Z,Z)-9,12-octadecadienal; (Z,Z)-11,13-octadecadienal; (E,E)-11,14-octadecadienal; (Z,Z)-13,15-octadecadienal; and (Z,Z,Z)-9,12,15-octadecatrienal.

In some embodiments, the aldehyde product is a pheromone or pheromone precursor. In some embodiments, the pheromone is selected from the group consisting of (Z)-7-hexadecenal, (Z)-9-hexadecenal, (Z)-11-hexadecenal, (Z)-9-octadecenal, Z-11-octadecenal, (Z)-13-octadecenal, (Z)-9-octadecenal, (E)-7-dodecenal, (E)-9-dodecenal, (E)-11-tetradecenal, (Z)-5-tetradecenal, (Z)-5-tetradecenal, (Z)-7-tetradecenal, (Z)-9-tetradecenal, (Z)-10-pentadecenal, (Z)-7-nonadecen-11-one, (Z)-11-tetradecenal, (E)-6-heneicosen-11-one, (4S,6S,7S)-4,6-dimethyl-7-hydroxynonan-3-one, (Z,Z)-9,11-hexadecadienal, (E,E)-10,12-hexadecadienal, (Z,E)-10,12-hexadecadienal, (E,E)-11,13-hexadecadienal, (E,E,Z)-4,6,11-hexadecatrienal, (Z,Z,E)-7,11,13-hexadecatrienal, (E,E,Z)-10,12,14-hexadecatrienal, (E,Z)-3,13-octadecadienal, (Z,Z)-13,15-octadecadienal, (Z,Z,Z)-9,12,15-octadecatrienal, (Z)-6,14-pentadecadienal, (Z)-9,13-tetradecadien-11-ynal, (Z)-13-hexadecen-11-ynal.

In some embodiments, the aldehyde product is selected from the group consisting of (Z)-hexadec-11-en-1-al, (Z)-hexadec-9-en-1-al, (Z)-octadec-13-en-1-al, (Z)-octadec-11-en-1-al, (Z)-octadec-9-en-1-al, and combinations thereof. In some embodiments, the aldehyde product is (Z)-hexadec-11-en-1-al.

In some embodiments, the ester is selected from the group consisting of a C₁₋₆ alkyl (Z)-hexadec-11-enoate, a C₁₋₆ alkyl (Z)-hexadec-9-enoate, a C₁₋₆ alkyl (Z)-octadec-11-enoate, a C₁₋₆ alkyl (Z)-octadec-13-enoate, a C₁₋₆ alkyl (Z)-octadec-9-enoate, and combinations thereof. In some embodiments, the ester is methyl (Z)-hexadec-11-enoate, methyl (Z)-hexadec-9-enoate, or methyl (Z)-octadec-9-enoate.

In some embodiments, the ester is selected from the group consisting of a triacyl glyceride (TAG) as in seed oils, where a composition of a mixture of triacyl glyceride (Z)-hexadec-11-enoate, triacyl glyceride (Z)-hexadec-9-enoate, triacyl glyceride (Z)-octadec-11-enoate, a triacyl glyceride (Z)-octadec-13-enoate, triacyl glyceride (Z)-octadec-9-enoate, and combinations thereof including triacyl glyceride of saturated palmitate and stearate. In some embodiments, the ester containing 1% molar to 100% molar concentrations of triacyl glyceride (Z)-hexadec-11-enoate, triacyl glyceride (Z)-hexadec-9-enoate, or triacyl glyceride (Z)-octadec-9-enoate.

In some embodiments, the preferred seed oils consist of the industrial seed oils Canola, Corn, Cottonseed, Soybean, Sunflower, Safflower, Grapeseed, Peanut, Palm, Palm kernal and Rice bran. Industrial seed oils are highly processed oils extracted from soybeans, corn, rapeseed (canola), cottonseed and sunflower and safflower seeds.

In some embodiments, unmatured fatty acid from various algae species may be the source of the whole algal biomass and crude algal oil from which the free fatty acids may be obtained. Algae are mostly aquatic photosynthetic organisms that range from microscopic flagellate to giant kelp. Algae may be loosely grouped into seven categories: Euglenophyta (euglenoids), Chrysophyta (golden-brown algae), Pyrrophyta (fire algae), Dinoflagellata, Chlorophyta (green algae), Rhodophyta (red algae), Paeophyta (brown algae), and Xanthophyta (yellow-green algae). Lipid extracted from any algae genus may be used in the various embodiments of the present invention, including Amphora, Anabaena, Anikstrodesmis, Botryococcus, Chaetoceros, Chlorella, Chlorococcum, Cyclotella, Cylindrotheca, Dunaliella, Emiliania, Euglena, Glossomastix, Haematococcus, Isochrysis, Monochrysis, Monoraphidium, Nannochloris, Nannochloropsis, Navicula, Nephrochloris, Nephroselmis, Nitzschia, Nodularia, Nostoc, Oochromonas, Oocystis, Oscillatoria, Pavlova, Phaeodactylum, Picochloris, Platymonas, Pleurochrysis, Porphyra, Pseudoanabaena, Pyramimonas, Scenedesmus, Stichococcus, Synechococcus, Synechocystis, Tetraselmis, Thalassiosira, and Trichodesmium.

In some instances, aldehyde products prepared according to the methods provided herein may contain small, characteristic amounts of isomeric components. The isomeric components may be present in amounts ranging from 0.001% to about 10% of the total aldehyde product. In some embodiments, the isomeric components are present in amounts of up to about 5% of the total aldehyde product. For example, (Z)-hexadec-11-en-1-al prepared according to the methods may contain isomeric (Z)-hexadec-9-en-1-al in an amount ranging from about 0.001% to about 5% of the total aldehyde product (e.g., about 0.1-5%, or 0.54%, or 1-4%, or about 3%).

### III. Examples

### Example 1. Preparation of (Z)-hexadec-11-en-1-al Via a Mixed Silyl Acetal

Methyl (Z)-hexadec-11-enoate (Z11-16ME, 1000 g, 3.725 mol; containing ca. 3% methyl (Z)-hexadec-11-enoate, Z9-16ME) and triethylsilane (476.5 g, 4.097 mol, 1.1 eq.) were added into a 5 L jacketed glass reactor, equipped with thermometer, dropping funnel, KPG stirrer, and bubbler. Before the addition of the borane catalyst, the mixture was stirred at 25° C. for 30 min. The (2-bromo-6-fluorophenyl)bis(2,3,5,6-tetrafluorophenyl)borane catalyst (1.8 g; 0.1 mol %) was dissolved in dry toluene (40 mL). The catalyst solution was slowly added to the reaction mixture. After the addition of 5 mL catalyst solution, intense gas formation was observed. The reaction temperature setpoint was 10° C. and the catalyst solution was slowly added keeping the reaction temperature below 52° C. After addition of approximately 20 mL of the catalyst solution into the reaction mixture, approx. 70 min after the start of the reaction, the gas formation became less intense, and the mixture was slowly cooled to 40° C. The reaction was stirred at room temperature and monitored by TLC. Another 5 mL portion of the catalyst solution was added to drive the reaction to completion. Qualitative analysis detected <0.5 area % Z11-16ME remaining, while showing 1.5 area % over-reduction. The reaction was quenched with 40 mL (0.41 mol) ethyl acetate and stirred for an additional 45 min. Hydrolysis was performed with by adding 1 L acetic acid and 250 mL water and stirring overnight at 30° C.

### Example 2. Preparation of (Z)-hexadec-11-en-1-al Via a Dialkyl Acetal and Vacuum Distillation

Borane catalyst (180 mg; 0.1 mol %) was dissolved in dry toluene (50 mL) and added into a 500 mL 3-neck round flask, equipped with thermometer, dropping funnel, and bubbler. To this solution was added dropwise a mixture of Z11-16ME (100 g, 0.372 mol; containing ca. 3% Z9-11ME) and triethylsilane (43.3 g, 0.372 mol) over a time period of 35 min. During the addition, the temperature rose to 37° C. and gas formation was limited to a manageable extent. The reaction was stirred at room temperature and monitored by TLC.

Qualitative analysis detected <0.5 area % Z11-16ME remaining, while showing <0.45 area % over-reduction. The reaction was quenched with 3 mL methyl acetate and stirred for an additional 45 min. The quenched mixture was diluted with 100 mL methanol containing 46 μL trimethylsilyl chloride to form the dimethyl acetal of the Z11-16 aldehyde. The mixture was stirred at room temperature overnight. The mixture was concentrated under vacuum and distilled on a packed column under vacuum. The relevant fraction was hydrolyzed by adding 1L acetic acid per 1 kg of recovered dimethyl acetal and 0.25 L 1M aq. HCl. The mixture was stirred at 40-45° C. for 2 hours. The final aldehyde was obtained in 52 mass % yield by diluting the mixture and extracting with MTBE.

### Example 3. Preparation of (Z)-hexadec-11-en-1-al Via a Mixed Acetal and Steam Distillation

Borane catalyst (180 mg, 0.1 mol %) was dissolved in dry toluene (50 mL) and added into a 500 mL 3-neck round flask, equipped with thermometer, dropping funnel, and bubbler. To this solution was added dropwise a mixture of Z11-16ME (100 g, 0.372 mol; containing ca. 3% Z9-16ME) and triethylsilane (43.3 g, 0.372 mol) over a time period of 35 min. During the addition the temperature rose to 37° C. and gas formation was limited to a manageable extent. The reaction was stirred at room temperature and monitored by TLC. Qualitative analysis detected <0.5 area % Z11-16ME remaining, while showing <1 area % over-reduction. The reaction was quenched with 3 mL methyl acetate and stirred for an additional 45 min. To the crude mixed acetal (183.1 g) was added a mixture of 100 mL acetic acid and 25 mL 1M HCl. The mixture was stirred at 45° C. for 2 hours. The organic phase was washed with water and the crude aldehyde extracted with MTBE. The product mixture was purified via counter current steam distillation to yield aldehyde in 66 mass %. Use of steam distillation provided a remarkable increase in isolated product as compared to vacuum distillation.

### Example 4. Preparation of (Z)-octadec-11-en-1-al Via a Mixed Silyl Acetal

Methyl (Z)-octadec-11-enoate (Z11-18ME) and triethylsilane (1.1 eq.) are added into a 5 L jacketed glass reactor, equipped with thermometer, dropping funnel, KPG stirrer, and bubbler. Before the addition of the borane catalyst, the mixture is stirred at 25° C. for 30 min. The (2-bromo-6-fluorophenyl)bis(2,4,6-trifluorophenyl)borane (0.1 mol %) is dissolved in dry toluene (40 mL). The catalyst solution is slowly added to the reaction mixture, maintaining the reaction temperature below 52° C. Approximately 60-70 min after the start of the reaction, the mixture is slowly cooled to 40° C. and monitored by TLC. After >99% consumption of starting material is observed, the reaction is quenched with ethyl acetate and stirred for an additional 45 min. Hydrolysis is performed with acetic acid and water as described in Example 1, and the product is isolated in good yield.

### Example 5. Preparation of (Z)-octadec-9-en-1-al Via a Dialkyl Acetal and Vacuum Distillation

(2-Chloro-6-fluorophenyl)bis(2,3,5,6-tetrafluorophenyl)borane catalyst (0.1 mol %) is dissolved in dry toluene (50 mL) and added into a 500 mL 3-neck round flask, equipped with thermometer, dropping funnel, and bubbler. To this solution is added dropwise a mixture of methyl (Z)-octadec-9-enoate (Z9-18ME, 0.372 mol) and triethylsilane (0.372 mol) over a time period of 30-40 min. During the addition, the temperature does not exceed 35-40° C. and gas is formed in controlled fashion. The reaction is stirred at room temperature and monitored by TLC. Following >99% consumption of the starting material, the reaction is quenched with methyl acetate and stirred for an additional 45-60 min. The quenched mixture is diluted with methanol containing trimethylsilyl chloride to form the dimethyl acetal of the Z9-18 aldehyde, and the mixture is stirred at room temperature overnight. The mixture is concentrated, distilled under vacuum, and hydrolyzed with acetic acid an aqueous hydrochloric acid as described in Example 2. The final aldehyde is isolated in good yield.

### Example 6. Preparation of (Z)-hexadec-9-en-1-al Via a Mixed Acetal and Steam Distillation

(Perfluoro-[1,1′-biphenyl]-2-yl)bis(2,3,5,6-tetrafluorophenyl)borane catalyst (0.1 mol %) is dissolved in dry toluene (50 mL) and added into a 500 mL 3-neck round flask, equipped with thermometer, dropping funnel, and bubbler. To this solution is added dropwise a mixture of methyl (Z)-hexadec-9-enoate (Z9-16ME, 0.372 mol) and triethylsilane (0.372 mol) over a time period of 30-45 min. During the addition the temperature does not exceed 35-40° C. and gas is generated in controlled fashion. The reaction is stirred at room temperature and monitored by TLC. After >99% consumption of the starting material is observed, the reaction is quenched with methyl acetate and stirred for an additional 45-60 min. To the crude mixed acetal is added a mixture of acetic acid and aqueous HCl as described in Example 3. The mixture is stirred at 45° C. for around 2 hours. The organic phase is washed with water and the crude aldehyde extracted with MTBE. The product mixture is purified via counter current steam distillation to yield the product aldehyde in good yield.

### Example 7. Preparation of (Z)-hexadec-11-en-1-al Via a Vacuum Distillation of the Dimethyl Acetal

To a clean, dry, nitrogen atmosphere 50 L glass-lined reactor equipped with a thermometer, addition tank port, reflux condenser and mechanical stirrer was added 10 kg Toluene and 11 g (22.8 mmol) (2-Bromo-6-fluorophenyl)-bis(2,3,5,6-tetrafluorophenyl) borane in 0.5 kg toluene, under nitrogen atmosphere. From the addition tank, was added the mixture of 13.9 kg (51.4 mol, >80% purity) Z11-16ME and 6.0 kg (51.9 mol) triethyl silane at a rate to maintain the temperature between 30° C. to 35° C., under nitrogen. After 1.5 hours of stirring at 35° C., IPC sample was taken for TLC and GC measurement. The reaction was complete by analysis, 250 g ethyl acetate was added to the mixture and was stirred for 1 hour. To the reactor was added 11.0 kg (343 mol) methanol and 10.2 g (9.4 mmol) trimethylsilyl chloride. The mixture was stirred for 14 hours at 25° C. After 14 hours, 20 g Na2CO3 was added and stirred for 30 mins. The reaction mixture was distilled under vacuo at 50° C. to remove excess methanol. MTBE (2.1 kg) and 2.8 kg deionized water were added and stirred for 10-15 minutes, the phases were allowed to separate, the aqueous layer was removed. The reaction mixture was washed with 2.8 kg deionized water by stirring for 15 minutes, the phases were allowed to separate, and the aqueous layer was removed. The reaction mixture was washed with 2.8 kg deionized water and 0.84 kg sodium chloride, as described above. After 10-15 minutes of stirring, the aqueous layer was removed. The organic layer was distilled under reduced pressure at 100° C. to remove solvent and water. After being cooled to 25° C., the reaction mixture was transferred to the high vacuum fractional distillation still. The first fraction contained 2.18 kg, Bpt 100° C. to 144° C. at 0.37 mbar of lights. The second fraction contained 12.16 kg, Bpt 160° C. to 165° C. at 0.37 mbar of Z11-16 dimethyl acetal and the distillation pot contained 0.45 kg of residue. The second fraction was used in the hydrolysis step. Fraction 2 (12.16 kg) was used for the hydrolysis of the Z11-16 dimethyl acetal to the Z11-16 aldehyde. To the clean, dry, nitrogen atmosphere 35 L glass-lined reactor was equipped with a thermometer, addition tank, reflux condenser and a mechanical stirrer was added 12.15 kg Z11-16 dimethyl acetal, under nitrogen atmosphere. To the reactor was added 12.8 kg Acetic acid and 3.33 kg 1M HCl, the reaction mixture was stirred for 6 hours at 40° C. (after every 2 hours sampling for IPC measurement). After 6 hour the reaction mixture was cooled to 25° C., and the aqueous layer was removed. Deionized water (3.3 kg) was added and stirred for 15 minutes, the organic and aqueous phases were allowed to separate, and the aqueous layer was removed. The reactor mixture was washed 2×3.3 kg Deionized water, as described above. The last wash was with 3.0 kg Deionized water and 0.35 kg NaHCO₃ for 15 minutes, the phases were allowed to separate, and the aqueous layer was removed. To the reaction mixture was added 4.5 kg MTBE, the MTBE was distilled under vacuo at 40° C. to 50° C. to azeotrope off water. The Z11-16 aldehyde from the reactor was drained into a clean, dry, nitrogen atmosphere HDPE drum, with 0.5 (w/w %) TBHQ and BHT as stabilizers. The final analysis was Z11-16 aldehyde (10.58 kg, 44.4 mol), GC analysis Z11-16 aldehyde 83.4 area % and overall yield of 86.4%.

### Example 8. Preparation of Z9-Octadecenal (Z9-18ald) Via Vacuum Distillation of the Dimethyl Acetal

To the clean, dry, nitrogen atmosphere 35 L glass-lined reactor equipped with a thermometer, addition tank, reflux condenser and mechanical stirrer was added 1.5 kg toluene and 4.9 g (2-Bromo-6-fluorophenyl)-bis(2,3,5,6-tetrafluorophenyl)borane in 0.5 kg toluene, under a nitrogen atmosphere. From the addition tank, a mixture of 6.0 kg Sunflower oil and 2.5 kg triethyl silane was added to the reactor at 30° C. to 35° C. over 30 minutes. After 1.5 hours of stirring IPC sample was taken for GC measurement, the reaction was complete. Ethyl acetate (110 g) was added to the reaction mixture and stirred for 30 minutes, followed by the additional of 4.8 kg methanol and 4.4 g trimethylsilyl chloride. The mixture was stirred for 18 hours at 25° C. After 18 hours, the lower glycerol containing layer was removed, followed by the addition of 8.6 g Na2CO3 and stirred for 30 mins. The excess methanol was removed under vacuo at 50° C., followed by the addition of 0.9 kg MTBE and 1.2 kg deionized water. The mixture was stirred for 15 minutes, the layers were allowed to separate, and the aqueous layer was removed. The organic phase was washed with 1.2 kg deionized water, removed, and washed with a mixture of 1.2 kg deionized water and 0.37 kg sodium chloride. After 15 minutes of stirring, the layers were allowed to separate, and the aqueous phase was removed. The organic phase was distilled under vacuo at 100° C. to remove water and MTBE. The pot residue (7.9 kg) was cooled to 25° C. and transferred to a vacuum distillation still. Vacuum distillation results were as follows: The first fraction contained 1.30 kg Bpt, 23° C. to 77° C. at 1.6 to 2.9 mbar. The second fraction contained 1.86 kg Bpt 110° C. to 135° C. at 2.2 to 2.3 mbar. The third fraction contained 3.28 kg Bpt 127° C. to 137° C. at 2.3 to 2.4 mbar. The fourth fraction contained 0.84 kg Bpt 137° C. to 185° C. at 0.9 to 1.5 mbar, pot contained 0.18 kg, and vacuum trap contained 0.82 kg of lights. To the clean, dry, nitrogen atmosphere 35 L glass-lined reactor equipped with a thermometer, addition tank, reflux condenser and mechanical stirrer was added 4.8 kg (Z9)-octadecenal dimethyl acetal (Z9-18DMA) (fractions 2 and 3 from distillation), under nitrogen atmosphere. To the reaction mixture was added 5.1 kg acetic acid and 1.4 kg 1M HCl and stirred at 40° C. for 5 hours. After 5 hours the mixture was cooled to 25° C. and 1.3 kg deionized water was added and stirred for 15 minutes. The phases were allowed to separate, and the aqueous layer was removed. The organic phase was washed 2× with 1.3 kg deionized water for 15 minutes, and the aqueous phases were removed. The last wash consisted of 1.2 kg deionized water and 0.13 kg NaHCO₃, stirred for 15 minutes, separated phases and the aqueous layer was removed. MTBE (1.9 kg) was added and distilled under vacuo at 45° C. to 50° C. to remove water and volatile solvents. The Z9-octadecanal was transferred to a clean, dry, nitrogen atmosphere HDPE drum containing 0.5 w/w % TBHQ and BHT. The final analysis indicated 4.0 kg Z9-18 aldehyde, GC purity of 83.3 area % and in an 79.38% overall yield.

### Example 9. Preparation of Z9-Hexadecenal (Z9-16ald) Via Vacuum Distillation of the Dimethyl Acetal

To the clean, dry, nitrogen atmosphere 35 L glassed-lined reactor equipped with a thermometer, addition tank, reflux condenser and mechanical stirrer was added 1.5 kg toluene and 4.5 g (2-bromo-6-fluorophenyl)-bis(2,3,5,6-tetrafluorophenyl) borane dissolved in 0.5 kg toluene, under nitrogen atmosphere. From the addition tank, the mixture of 5.0 kg methyl Z9-hexadecenoate (Z9-16ME) and 2.25 kg triethyl silane was added at 30° C. to 35° C. Reduction of Z9-16FAME with triethyl silane was exothermic, external cooling was required. During the reaction the internal temperature was maintained between 30° C. and 35° C. by external cooling. After the reagents were added, the mixture was stirred for 45 minutes and IPC sample was taken out for TLC and GC measurements. Ethyl acetate (180 g) was added and stirred for 30 mins to decompose the unreacted triethyl silane. To the crude mixed acetal was added 4.0 kg methanol and 2.06 g trimethylsilyl chloride, the mixture was stirred at 40° C. for 4 hours. After 4 hours, IPC measurement, TLC indicated complete formation of Z9-16 dimethyl acetal (Z9-16DMA), followed by the addition of 0.5 kg Na2CO3 and stirred for 30 mins. The mixture was distilled under vacuo at 40° C. until removal of excess methanol. Tert-Butyl methyl ether (TBME, 3.7 kg) and 2.5 kg deionized were added and stirred for 15 minutes, the phases were allowed to separate and the aqueous layer was removed. The organic layer was distilled under vacuo at 40° C. to remove water and TBME. The reaction mixture was transferred to a vacuum distillation still. Distillation results are below:

| | 1^{st} | 2^{nd} | 3^{rd} | 4^{th} | 5^{th} | 6^{th} | Pot |
|---|---|---|---|---|---|---|---|
| | fraction | fraction | fraction | fraction | fraction | fraction | Residue |
| mass(kg) | 1.80 | 0.83 | 1.65 | 1.70 | 1.04 | 0.28 | 0.58 |
| T_{head} ° C. | 28 | 65-160 | 150-173 | 161-182 | 166-183 | 161-163 | |
| Tₕₑₐₜₑᵣ ° C. | 118 | 190-276 | 276-278 | 212-277 | 277-279 | 260 | |
| pressure | 5.2 | 3.4-6.0 | 3.3-4.5 | 3.5-3.6 | 3.4-3.6 | 2.7 | |
| mbar | | | | | | | |

Fractions 3 through 6 (4.7 kg Z9-16DMA) were combined and added to a clean, dry, nitrogen atmosphere 35 L glassed-lined reactor equipped with a thermometer, addition tank, reflux condenser and mechanical stirrer, under nitrogen atmosphere. Followed by the addition of 9.9 kg acetic acid and 2.5 kg 1M HCl, the mixture as stirred at 40° C. for 1 hour, after 1 hour IPC measurement, GC, complete hydrolysis of the acetal was detected, level of Z9-16DMA was <1%. After 1 hour the mixture was cooled to 25° C. and 2.5 kg of deionized water was added and stirred for 15 minutes. The phases were allowed to separate, and the aqueous layer was removed. The organic phase was washed 2× with 2.5 kg of deionized water and stirred for 15 minutes. Next, the phases separated, and the aqueous layers were removed. The last wash consisted of 2.5 kg deionized water and 0.24 kg sodium bicarbonate, stirred for 15 minutes, phases were allowed to separate and the aqueous layer was removed. To the Z9-hexadecenal was added 3.5 kg MTBE and distilled water under vacuo at 40° C. to 50° C. to remove water and TBME. The Z9-hexadecenal was transferred to a clean, dry HDPE drum, containing 0.5 w/w % TBHQ and BHT.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A method for preparing an aldehyde product, the method comprising:
forming a reaction mixture comprising a silane, a borane catalyst, and an ester according to Formula III: 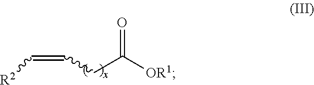
maintaining the reaction mixture under conditions for forming a mixed acetal according to Formula II: 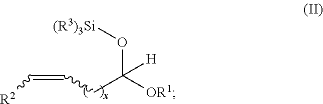
and
converting the mixed acetal to an aldehyde according to Formula I: 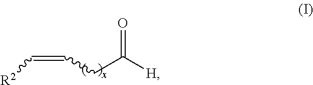
thereby forming the aldehyde product;
wherein:
subscript x is an integer ranging from 0 to 17,
R¹ is C₁₋₈ alkyl or glycerol, and
R² is selected from the group consisting of C₁₋₁₈ alkyl and C₁₋₁₈ alkenyl,
each R³ is independently selected from the group consisting of C₁₋₈ alkyl and C₆₋₁₄ aryl.

2. The method of claim 1, wherein:
the ester is an ester according to Formula IIIa or a triacyl glyceride (TAG) according to Formula IIIc: 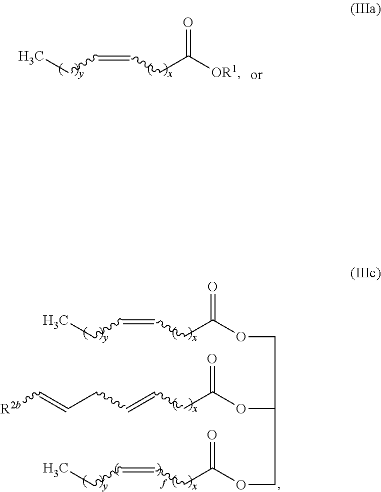
wherein:
f is 0 or 1,
R^{2b} is C₁₋₁₄ alkyl,
the mixed acetal is a mixed acetal according to Formula IIa: 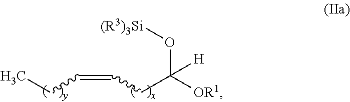
and
the aldehyde product is an aldehyde according to Formula Ia: 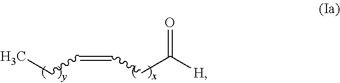
wherein subscript y is an integer ranging from 0 to 17.

3. The method of claim 1 or claim 2, wherein forming the reaction mixture comprises:
(a) combining the ester with the silane; and
(b) adding the borane catalyst to the mixture resulting from step (a).

4. The method of claim 1 or claim 2, wherein forming the reaction mixture comprises combining the ester with a first portion of the silane to form a first mixture.

5. The method of claim 4, wherein the first portion of the silane constitutes from about 1% to about 25% of the total silane.

6. The method of claim 4 or claim 5, wherein all of the ester is combined with the first portion of silane in the first mixture.

7. The method of claim 4 or claim 5, wherein a first portion of the ester is combined with the first portion of silane in the first mixture.

8. The method of claim 7, wherein the first portion of the ester constitutes from about 1% to about 25% of the total ester.

9. The method of any one of claims 4-8, wherein the borane catalyst is added to the first mixture to form a second mixture.

10. The method of claim 9, further comprising adding a second portion of the silane to the second mixture.

11. The method of claim 9 or claim 10, further comprising adding a second portion of the ester to the second mixture.

12. The method of claim 11, wherein the second portion of the ester is added to the second mixture concomitantly with the second portion of the silane.

13. The method of any one of claims 1-12, wherein the reaction mixture is maintained at a temperature ranging from about 35° C. to about 45° C.

14. The method of any one of claims 1-13, wherein converting the mixed acetal to the aldehyde comprises hydrolyzing the mixed acetal.

15. The method of any one of claims 1-13, wherein converting the mixed acetal to the aldehyde comprises converting the mixed acetal to a dialkyl acetal and hydrolyzing the dialkyl acetal.

16. The method of claim 14 or claim 15, wherein hydrolyzing the mixed acetal or the dialkyl acetal comprises combining the mixed acetal or the dialkyl acetal with acetic acid and hydrochloric acid.

17. The method of any one of claims 1-16, further comprising removing silanol and disiloxane byproducts produced during hydrolysis of the mixed acetal.

18. The method of claim 17, wherein the byproducts are removed via vacuum distillation or steam distillation.

19. The method of any one of claims 1-18, wherein the silane is triethylsilane.

20. The method of any one of claims 1-19, wherein the borane catalyst is a compound according to Formula IV: 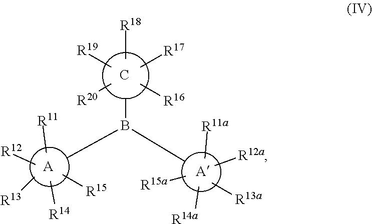
wherein
B is boron;
the A ring and the A′ ring are aryl groups; wherein
R¹¹ and R^{11a} are independently selected from groups having small steric demand, preferably H, D and F;
R¹⁵ and R^{15a} are independently selected from groups having small steric demand, preferably H, D and F;
R¹², R¹³, R¹⁴, R^{12a}, R^{13a} and R^{14a} are independently selected from the group consisting of H, D, F, Cl, Br, I, SF₅, alkyl, cycloalkyl, alkenyl, cycloalkenyl, aryl and heteroaryl, wherein alkyl, cycloalkyl, alkenyl, cycloalkenyl, aryl, and heteroaryl are optionally substituted;
the C ring is aryl group, wherein
R¹⁶ is selected from groups having small steric demand, preferably H, D and F;
R²⁰ is selected from groups having large steric demand, preferably from the group consisting of Cl, Br, I, SF₅, alkyl, cycloalkyl, alkenyl, cycloalkenyl, aryl and heteroaryl, wherein alkyl, cycloalkyl, alkenyl, cycloalkenyl, aryl and heteroaryl are optionally substituted;
R¹⁷, R¹⁸ and R¹⁹ are independently selected from the group consisting of H, D, F, Cl, Br, I, SF₅, alkyl, cycloalkyl, alkenyl, cycloalkenyl, aryl and heteroaryl, where alkyl, cycloalkyl, alkenyl, cycloalkenyl, aryl and heteroaryl are optionally substituted.

21. The method of claim 20, wherein R²⁰ is not a pentafluorophenyl group when R¹¹-R¹⁵, R^{11a}-R^{15a}, and R¹⁶-R¹⁹ are F.

22. The method of claim 20 or claim 21, wherein the catalyst has a structure according to Formula IVa: 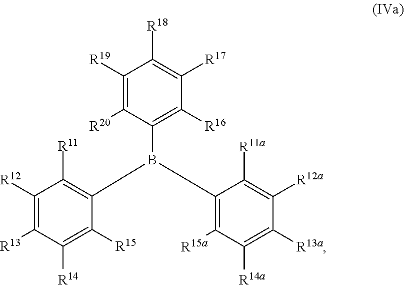
wherein
R¹¹ and R^{11a} are independently selected from the group consisting of H, D and F;
R¹⁵ and R^{15a} are independently selected from the group consisting of H, D and F;
R¹², R¹³, R¹⁴, R^{12a}, R^{13a} and R^{14a} are independently selected from the group consisting of H, D, F, Cl, Br, alkyl, cycloalkyl and aryl, wherein alkyl, cycloalkyl and aryl are optionally substituted;
R¹⁶ is selected from the group consisting of H, D and F;
R²⁰ is selected from the group consisting of Cl, Br, I, SF₅, alkyl, cycloalkyl and aryl, wherein alkyl, cycloalkyl and aryl are optionally substituted;
R₇, R₈ and R₉ are independently selected from the group consisting of H, D, F, Cl, Br, alkyl and cycloalkyl, wherein alkyl and cycloalkyl are optionally substituted.

23. The method of claim 28, wherein:
R¹¹, R^{11a}, R¹⁵ and R^{15a} are F;
R¹², R¹³, R¹⁴, R^{12a}, R^{13a}, and R^{14a} are independently selected from H and F,
R¹⁶, R¹⁷, R¹⁸ and R¹⁹ are independently selected from H and F; and
R²⁰ is selected from C₁, Br, and pentafluorophenyl.

24. The method of claim 20 or claim 21, wherein the A ring and the A′ ring are independently selected from the group consisting of pentafluorophenyl, 2,3,4,6-tetrafluorophenyl, 2,4,6-trifluorophenyl, and 2,3,6-trifluorophenyl.

25. The method of any one of claims 20, 21, and 24, wherein the C ring is selected from the group consisting of 2-chloro-6-fluorophenyl, 2-bromo-6-fluorophenyl, and perfluoro-1,1′-biphen-2-yl.

26. The method of any one of claims 20-25, wherein the catalyst is selected from the group consisting of:

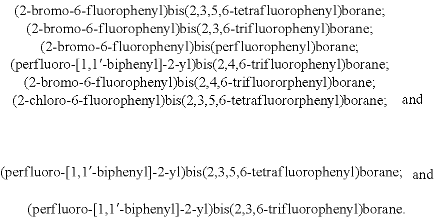

27. The method of any one of claims 1-26, wherein the aldehyde product is selected from the group consisting of (Z)-hexadec-11-en-1-al, (Z)-hexadec-9-en-1-al, (Z)-octadec-13-en-1-al, (Z)-octadec-11-en-1-al, (Z)-octadec-9-en-1-al, and combinations thereof.

28. The method of any one of claim 27, wherein the aldehyde product is selected from the group consisting of (Z)-hexadec-1 i-en-1-al, (Z)-hexadec-9-en-1-al, and (Z)-octadec-9-en-1-al.

29. The method of any one of claims 1-28, wherein the ester is selected from the group consisting of a C₁₋₆ alkyl (Z)-hexadec-11-enoate, a C₁₋₆ alkyl (Z)-hexadec-9-enoate, a C₁₋₆ alkyl (Z)-octadec-13-enoate, a C₁₋₆ alkyl (Z)-octadec-11-enoate, a C₁₋₆ alkyl (Z)-octadec-9-enoate, and combinations thereof.

30. The method of claim 29, wherein the ester is selected from the group consisting of methyl (Z)-hexadec-11-enoate, methyl (Z)-hexadec-9-enoate, and methyl (Z)-octadec-9-enoate.

31. An aldehyde product prepared according to the method of any one of claims 1-30.

32. The aldehyde product of claim 31, comprising (Z)-hexadec-11-en-1-al and (Z)-hexadec-9-en-1-al, wherein the (Z)-hexadec-9-en-1-al is present in an amount equal to or less than 5% of the total aldehyde product.

33. The method of claim 1, wherein:
the ester is an ester according to Formula IIIb: 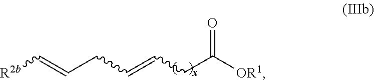
wherein the mixed acetal is a mixed acetal according to Formula IIb: 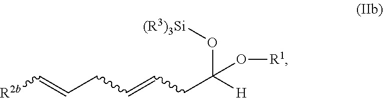
and
the aldehyde product is an aldehyde according to Formula Ib: 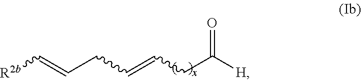
wherein R^{2b} is C₁₋₁₄ alkyl and wherein x is an integer ranging from 0 to 17.

34. The method of claim 33, wherein the aldehyde according to Formula Ib is (Z,Z)-9,12-octadecadienal.

35. The method of claim 34, wherein x is 7.

36. The method of claim 33, wherein forming the reaction mixture comprises:
(a) combining the ester with the silane; and
(b) adding the borane catalyst to the mixture resulting from step (a).

37. The method of claim 33, wherein forming the reaction mixture comprises combining the ester with a first portion of the silane to form a first mixture.
